(19) United States
(12) Patent Application Publication
(10) Pub. No.: US 2019/0142753 A1
(43) Pub. Date: May 16, 2019

(54) METHOD OF PREPARING PHARMACEUTICAL MICROSPHERE

(71) Applicant: Zhongshuai Pharmaceutical Sci & Tech Co., Ltd, Zhengzhou (CN)

(72) Inventors: Weidong SUN, Zhengzhou (CN); Yongsheng DONG, Zhengzhou (CN); Rui LI, Zhengzhou (CN); Li MA, Zhengzhou (CN)

(21) Appl. No.: 16/247,605

(22) Filed: Jan. 15, 2019

Related U.S. Application Data
(63) Continuation-in-part of application No. PCT/CN2017/070891, filed on Jan. 11, 2017.

(30) Foreign Application Priority Data
Jul. 15, 2016 (CN) 201610556389.7

(51) Int. Cl. A61K 9/16; A61K 9/50

(52) U.S. Cl. CPC A61K 9/1676; A61K 9/5073; A61K 9/5089

(57) ABSTRACT

A method of preparing a pharmaceutical microsphere, the method including: adding a silica microsphere to an acid, stirring, adding an alkali to neutralize the acid until the pH value of the mixture of the acid and the alkali is 6-7, filtering and collecting a resulting product, washing, drying, and sieving, to yield a pharmaceutical microsphere. Also provided is a method of preparing a pharmaceutical preparation includes coating the pharmaceutical microsphere with an isolation layer, a taste-masking layer, a sustained-release layer, or a combination thereof.

### CROSS-REFERENCE TO RELAYED APPLICATIONS

This application is a continuation-in-part of International Patent Application No. PCT/CN2017/070891 with an international filing date of Jan. 11, 2017, designating the United States, now pending, and further claims foreign priority benefits to Chinese Patent Application No. 201610556389.7 filed Jul. 15, 2016. The contents of all of the aforementioned applications, including any intervening amendments thereto, are incorporated herein by reference. Inquiries from the public to applicants or assignees concerning this document or the related applications should be directed to: Matthias Scholl P C., Attn.: Dr. Matthias Scholl Esq., 245 First Street, 18th Floor, Cambridge, Mass. 02142.

### BACKGROUND

This disclosure relates to a method of preparing a pharmaceutical microsphere.

Conventional blank pharmaceutical pellets include sugar pellets, microcrystalline cellulose pellets, starch pellets, lactose pellets, silica pellets, and the like. The blank pellets can be coated with active pharmaceutical ingredients using Wuster coating technology to yield pharmaceutical preparations in pellet form. In general, the microspherical preparations have a particle size of between 300 and 800 μm. For children, the elderly, and patients with dysphagia, the preparations are too large to swallow.

### SUMMARY

Disclosed is a method of preparing a pharmaceutical microsphere. The prepared pharmaceutical microsphere is efficient in producing microspherical preparations with particle sizes of no more than 150 μm.

The disclosure provides a method of preparing a pharmaceutical microsphere, the method comprising: adding a silica microsphere to an acid, stirring, adding an alkali to neutralize the acid until a pH value of a mixture of the acid and the alkali is 6-7, filtering and collecting a resulting product, washing, drying, and sieving, to yield a pharmaceutical microsphere.

The method can further comprise heating glass powders in a beading furnace to a temperature of 600-1200° C., and then cooling the glass powders to a temperature of 100-300° C., to yield the silica microsphere.

The method can further comprise grinding a rock crystal comprising at least 90.0 wt. % of silica to yield the silica microsphere.

The acid can be selected from sulfuric acid, hydrochloric acid, phosphoric acid, nitric acid, or a mixture thereof, and has a concentration of 1-10 wt. %; and the base can be selected from sodium hydroxide, potassium hydroxide, or a mixture thereof.

Specifically, the method can comprise: preparing the silica microsphere in a beading furnace or grinding a rock crystal to yield the silica microsphere, adding the silica microsphere to a 5-10% hydrochloric acid solution, stirring for 1-2 hours, allowing for standing, removing a supernatant, adding a 1-5 mol/L sodium hydroxide aqueous solution to neutralize the hydrochloric acid solution until a pH value thereof is 7, filtering and collecting the resulting product, washing with purified water, drying, and sieving, to yield the pharmaceutical microsphere.

Specifically, the method can comprise: preparing the silica microsphere in a beading furnace or grinding a rock crystal comprising 99.0 wt. % of silica to yield the silica microsphere having 80 meshes, adding the silica microsphere to a 10% hydrochloric acid solution, stirring for 1 hour, allowing for standing, removing a supernatant, adding a 1 mol/L sodium hydroxide aqueous solution to neutralize the hydrochloric acid solution until a pH value thereof is 7, filtering and collecting the resulting product, washing with purified water, drying, and sieving, to yield a pharmaceutical microsphere having 80 meshes.

Specifically, the method can comprise: preparing the silica microsphere in a beading furnace or grinding a rock crystal comprising 99.9 wt. % of silica to yield the silica microsphere having 120 meshes, adding the silica microsphere to a 10% hydrochloric acid solution, stirring for 1 hour, allowing for standing, removing a supernatant, adding a 1 mol/L sodium hydroxide aqueous solution to neutralize the hydrochloric acid solution until a pH value thereof is 7, filtering and collecting the resulting product, washing with purified water, drying, and sieving, to yield a pharmaceutical microsphere having 120 meshes.

The particle size of the pharmaceutical microsphere can range from 40 to 250 μm; the bulk density of the pharmaceutical microsphere can range from 1.2 to 1.6 g/cm³, and the roundness of the pharmaceutical microsphere can be between 10-12°.

The particle size of the pharmaceutical microsphere can range from 50 to 100 μm.

The disclosure further provides a method of preparing a pharmaceutical preparation, the method comprising: adding a silica microsphere to an acid, stirring, adding an alkali to neutralize the acid until a pH value of a mixture of the acid and the alkali is 6-7, filtering and collecting a resulting product, washing, drying, and sieving, to yield a pharmaceutical microsphere, and coating the pharmaceutical microsphere.

The pharmaceutical preparation can have a particle size of no more than 150 μm.

The yield ratio of coating the pharmaceutical microsphere can be more than 90%.

The pharmaceutical microsphere can be coated by an isolation layer, a taste-masking layer, a sustained-release layer, or a combination thereof.

Advantages of the method of preparing a pharmaceutical microsphere as described in the disclosure are summarized as follows.

1. The produced pharmaceutical microsphere of silica is non-toxic, and is compatible with the human body.

2. The roundness of the pharmaceutical microsphere is excellent, improving the coating efficiency.

3. The produced pharmaceutical microsphere exhibits relatively high mechanical strength.

4. The particle size of the pharmaceutical preparations prepared with the pharmaceutical microsphere as a blank pellet can be less than 150 μm.

### DETAILED DESCRIPTION

To further illustrate, embodiments detailing a method of preparing a pharmaceutical microsphere are described below. It should be noted that the following embodiments are intended to describe and not to limit the disclosure.

### Example 1: Preparation of Silica Microspheres

Prepare silica microspheres in a beading furnace or by grinding a rock crystal comprising at least 99.0 wt. % of silica using a ball mill. 1 kg of the silica microspheres (80 meshes) was added to 10% hydrochloric acid solution, stirred for one hour, and allowed to stand. The supernatant was removed, and 1 mol/L sodium hydroxide aqueous solution was added to the concentrate to adjust the pH value thereof to be 7. The concentrate was filtered, and the resulting product was washed with purified water, dried, and sieved to yield 80 meshes of silica microspheres.

### Example 2: Preparation of Silica Microsphere

Prepare silica microspheres in a beading furnace or by grinding a rock crystal comprising at least 99.9 wt. % of silica using a ball mill. 1 kg of the silica microspheres (100-120 meshes) was added to 10% hydrochloric acid solution, stirred for one hour, and allowed to stand. The supernatant was removed, and 1 mol/L sodium hydroxide aqueous solution was added to the concentrate to adjust the pH value thereof to be 7. The concentrate was filtered, and the resulting product was washed with purified water, dried, and sieved to yield 100-120 meshes of silica microspheres.

### Example 3: Preparation of Azithromycin for Suspension

### 1. Preparation of Azithromycin-Containing Pharmaceutical Microsphere

1.1) Materials: 300 g of silica microsphere having a particle size of 0.075-0.10 mm, 150 g of azithromycin (100 meshes), 10 g of hydroxy propyl methyl cellulose (HPMC, E5), 5 g of talcum powder, and 2000 mL of 85% alcohol.

1.2) Preparation of pharmaceutical solution: 10 g of hydroxy propyl methyl cellulose (HPMC, E5) was added to 2000 mL of 85% alcohol, stirred, and dissolved to yield a clear solution. 150 g of azithromycin was added to the clear solution, stirred for an hour, followed by addition of 5 g of talcum powder, stirred for 15 min, to yield a pharmaceutical solution.

1.3) 300 g of silica microsphere (150-200 meshes) having a particle size of 0.075-0.10 mm was added to a fluid-bed coater with a bottom spray gun. The silica microsphere was coated with the pharmaceutical solution by the bottom spray gun. The blowing frequency was 25 Hz, the inlet temperature was 45° C., the diameter of the spraying nozzle was 1.0 mm, the atomization pressure was 0.15 megapascal, and the pumping speed of peristaltic pump was 5 rpm. When the silica microsphere temperature reached 35° C., start the peristaltic pump to pump the pharmaceutical solution. After the coating was finished, stop the peristaltic pump. 20 min later, the silica microsphere was taken out, placed in a 50° C. air dry oven for 2 hours, sieved, to yield an azithromycin-containing pharmaceutical microsphere.

### 2. Preparation of Taste-Masking Azithromycin-Containing Pharmaceutical Microsphere

2.1) Coating an Isolation Layer on the Azithromycin-Containing Pharmaceutical Microsphere

20 g of HPMC was added to 500 mL of purified water, stirred for dissolution, to yield a clear solution.

The azithromycin-containing pharmaceutical microsphere in prescription dosage was added to a fluid-bed coater with a bottom spray gun. The pharmaceutical microsphere was coated with the clear solution by the bottom spray gun. The blowing frequency was 25 Hz, the inlet temperature was 45° C., the diameter of the spraying nozzle was 1.0 mm, the atomization pressure was 0.16 megapascal, and the pumping speed of the peristaltic pump was 5 rpm. When the pharmaceutical microsphere temperature reached 40° C., start the peristaltic pump to pump the clear solution. After the coating was finished, stop the peristaltic pump. 20 min later, the pharmaceutical microsphere was taken out, placed in a 50° C. air dry oven for 2 hours, sieved, to yield an azithromycin-containing pharmaceutical microsphere comprising an isolation layer.

2.2) Coating a Taste-Masking Layer on the Azithromycin-Containing Pharmaceutical Microsphere

Preparation of a Taste-Masking Solution

150 g of eudragit E100, 15 g of triethyl citrate, and 1500 mL of 90% alcohol solution were mixed, followed by addition of 15 g of talcum powder, to yield a taste-masking solution.

Preparation of a Taste-Masking Pharmaceutical Microsphere

The azithromycin-containing pharmaceutical microsphere comprising an isolation layer was added to a fluid-bed coater with a bottom spray gun. The pharmaceutical microsphere was coated with the taste-masking solution by the bottom spray gun. The blowing frequency was 25 Hz, the inlet temperature was 45° C., the diameter of the spraying nozzle was 1.0 mm, the atomization pressure was 0.15 megapascal, and the pumping speed of peristaltic pump was 5 rpm. When the pharmaceutical microsphere temperature reached 40° C., start the peristaltic pump to pump the taste-masking solution. After the coating was finished, stop the peristaltic pump. 20 min later, the pharmaceutical microsphere was taken out, placed in a 50° C. air dry oven for 2 hours, sieved using 100 and 120 meshes sieves, to yield a taste-masking azithromycin-containing pharmaceutical microsphere.

### 3. Preparation of Azithromycin for Suspension

The taste-masking azithromycin-containing pharmaceutical microsphere, 10 g of talcum powder, 30 g of mannitol, 20 g of fruit flavor, 20 g of sodium benzoate, and 40 g of xanthan gum were evenly mixed, qualified, to yield azithromycin for suspension.

### Example 4: Preparation of Sustained-Release Capsule of Cefprozil

### 1. Preparation of Cefprozil-Containing Pharmaceutical Microsphere

1.1) Materials: 200 g of silica microsphere having a particle size of 0.12-0.15 mm, 125 g of cefprozil (10 μm), 20 g of povidone, 5 g of talcum powder, and 2000 mL of 60% alcohol.

1.2) Preparation of pharmaceutical solution: 20 g of povidone was added to 2000 mL of 85% alcohol, stirred, and dissolved to yield a clear solution. 125 g of cefprozil was added to the clear solution, stirred for an hour, followed by addition of 5 g of talcum powder, stirred for 15 min, to yield a pharmaceutical solution.

1.3) 200 g of silica microsphere (100-120 meshes) having a particle size of 0.12-0.15 mm was added to a fluid-bed coater with a bottom spray gun. The silica microsphere was coated with the pharmaceutical solution by the bottom spray gun. The blowing frequency was 20 Hz, the inlet temperature was 45° C., the diameter of the spraying nozzle was 1.0 mm, the atomization pressure was 0.15 megapascal, and the pumping speed of peristaltic pump was 5 rpm. When the silica microsphere temperature reached 30° C., start the peristaltic pump to pump the pharmaceutical solution. After the coating was finished, stop the peristaltic pump. 20 min later, the silica microsphere was taken out, placed in a 50° C. air dry oven for 2 hours, sieved, to yield a cefprozil-containing pharmaceutical microsphere.

### 2. Preparation of Sustained-Release Microsphere of Cefprozil

2.1) Coating an Isolation Layer on the Cefprozil-Containing Pharmaceutical Microsphere

20 g of HPMC was added to 400 mL of purified water, stirred for dissolution, to yield a clear solution.

The cefprozil-containing pharmaceutical microsphere in prescription dosage was added to a fluid-bed coater with a bottom spray gun. The pharmaceutical microsphere was coated with the clear solution by the bottom spray gun. The blowing frequency was 20 Hz, the inlet temperature was 45° C., the diameter of the spraying nozzle was 1.0 mm, the atomization pressure was 0.16 megapascal, and the pumping speed of peristaltic pump was 6 rpm. When the pharmaceutical microsphere temperature reached 40° C., start the peristaltic pump to pump the clear solution. After the coating was finished, stop the peristaltic pump. 15 min later, the pharmaceutical microsphere was taken out, placed in a 50° C. air dry oven for 2 hours, sieved, to yield a cefprozil-containing pharmaceutical microsphere comprising an isolation layer.

2.2) Coating a Sustained-Release Layer on the Cefprozil-Containing Pharmaceutical Microsphere

Preparation of a Sustained-Release Solution

50 g of eudragit RS100, 5 g of triethyl citrate, and 400 mL of 90% alcohol solution were mixed, followed by addition of 15 g of talcum powder, to yield a sustained-release solution.

Preparation of a Sustained-Release Pharmaceutical Microsphere

The cefprozil-containing pharmaceutical microsphere comprising an isolation layer was added to a fluid-bed coater with a bottom spray gun. The pharmaceutical microsphere was coated with the taste-masking solution by the bottom spray gun. The blowing frequency was 20 Hz, the inlet temperature was 45° C., the diameter of the spraying nozzle was 1.0 mm, the atomization pressure was 0.15 megapascal, and the pumping speed of peristaltic pump was 5 rpm. When the pharmaceutical microsphere temperature reached 40° C., start the peristaltic pump to pump the sustained-release solution. After the coating was finished, stop the peristaltic pump. 20 min later, the pharmaceutical microsphere was taken out, placed in a 50° C. air dry oven for 2 hours, sieved using 80 and 110 meshes sieves, to yield a sustained-release cefprozil-containing pharmaceutical microsphere.

### 3. Preparation of Sustained-Release Capsule of Cefprozil

The sustained-release cefprozil-containing pharmaceutical microsphere, 8 g of talcum powder, 10 g of mannitol, and 15 g of fruit flavor were evenly mixed, qualified, and packaged, to yield a sustained-release capsule of cefprozil. Prior to use, open the capsule, the active ingredients can be administered with cheese, pudding, jam and other foods.

### Example 5: Preparation of Roxithromycin for Suspension

### 1. Preparation of Roxithromycin-Containing Pharmaceutical Microsphere

1.1) Materials: 200 g of silica microsphere having a particle size of 0.10-0.12 mm, 100 g of roxithromycin (100 meshes), 35 g of hydroxy propyl methyl cellulose (HPMC), 5 g of talcum powder, and 2000 mL of 70% alcohol.

1.2) Preparation of pharmaceutical solution: 35 g of hydroxy propyl methyl cellulose (HPMC) was added to 2000 mL of 70% alcohol, stirred, and dissolved to yield a clear solution. 100 g of roxithromycin (100 meshes) was added to the clear solution, stirred for an hour, followed by addition of 5 g of talcum powder, stirred for 15 min, to yield a pharmaceutical solution.

1.3) 200 g of silica microsphere (130-150 meshes) having a particle size of 0.10-0.12 mm was added to a centrifugal coater. The centrifugal rotational speed was 200-300 rpm. The blowing frequency was 5-50 Hz, the inlet temperature was 40-60° C., the diameter of the spraying nozzle was 0.8 mm, the atomization pressure was 0.01-0.05 megapascal, and the pumping speed of peristaltic pump was 5-15 rpm. Start the peristaltic pump to pump the pharmaceutical solution, when the surface of the silica microsphere was wet, start the automatic feeding device. The frequency of the feeder was 20 rpm. After the coating was finished, stop the peristaltic pump. 10-30 min later, the silica microsphere was taken out, placed in a 50° C. air dry oven for 2-6 hours, sieved, to yield a roxithromycin-containing pharmaceutical microsphere.

### 2. Preparation of Taste-Masking Roxithromycin-Containing Pharmaceutical Microsphere

2.1) Coating an Isolation Layer on the Roxithromycin-Containing Pharmaceutical Microsphere

20 g of HPMC was added to 400 mL of purified water, stirred for dissolution, followed by addition of 5 g of talcum powder, to yield a clear solution.

The roxithromycin-containing pharmaceutical microsphere was added to a coater. The diameter of the nozzle was 1.0 mm, the atomization pressure was 1.4-2.0 bar, the pressure of the needle valve was 4.0 bar, the temperature of the inlet air was 40-60° C., the temperature of the outlet air was 30-50° C., the rotation speed of the main engine was 10-25 rpm, the inlet air volume was 500-900 m³/h, the outlet air volume was 1000-2900 m³/h, and the negative pressure of the boiler was about −0.1 kPa. When the pharmaceutical microsphere temperature reached 30-45° C., start the peristaltic pump to pump the clear solution. The pumping speed of the peristaltic pump was 5-25 rpm. After the coating was finished, stop the peristaltic pump. The pharmaceutical microsphere was taken out, placed in a 50° C. air dry oven for 2-6 hours, sieved, to yield a roxithromycin-containing pharmaceutical microsphere comprising an isolation layer.

2.2) Coating a Taste-Masking Layer on the Roxithromycin-Containing Pharmaceutical Microsphere

Preparation of a Taste-Masking Solution

50 g of polyacrylic resin IV and 400 mL of 95% alcohol solution were mixed, followed by addition of 6 g of talcum powder, to yield a taste-masking solution.

Preparation of a Taste-Masking Pharmaceutical Microsphere

The roxithromycin-containing pharmaceutical microsphere comprising an isolation layer was added to a coater. The diameter of the nozzle was 1.0 mm, the atomization pressure was 1.4-2.0 bar, the pressure of the needle valve was 4.0 bar, the temperature of the inlet air was 40-60° C., the temperature of the outlet air was 30-50° C., the rotation speed of the main engine was 10-25 rpm, the inlet air volume was 500-900 m³/h, the outlet air volume was 1000-2900 m³/h, and the negative pressure of the boiler was about −0.1 kPa. When the pharmaceutical microsphere temperature reached 30-45° C., start the peristaltic pump to pump the taste-masking solution. The pumping speed of the peristaltic pump was 5-25 rpm. After the coating was finished, stop the peristaltic pump. The pharmaceutical microsphere was taken out, placed in a 50° C. air dry oven for 2-6 hours, sieved using 80 and 120 meshes sieves, to yield a taste-masking roxithromycin-containing pharmaceutical microsphere.

### 3. Preparation of Roxithromycin for Suspension

The taste-masking roxithromycin-containing pharmaceutical microsphere, 5 g of talcum powder, 10 g of mannitol, 5 g of fruit flavor, 10 g of sodium benzoate, and 20 g of xanthan gum were evenly mixed, qualified, to yield roxithromycin for suspension.

### Example 6: Preparation of Lincomycin Dry Suspension

### 1. Preparation of Lincomycin-Containing Pharmaceutical Microsphere

1.1) Materials: 200 g of silica microsphere having a particle size of 0.10-0.12 mm, 150 g of lincomycin (100 meshes), 20 g of povidone, 5 g of talcum powder, and 2000 mL of 60% alcohol.

1.2) Preparation of pharmaceutical solution: 20 g of povidone was added to 2000 mL of 60% alcohol, stirred, and dissolved to yield a clear solution. 150 g of lincomycin (100 meshes) was added to the clear solution, stirred for an hour, followed by addition of 5 g of talcum powder, stirred for 15 min, to yield a pharmaceutical solution.

1.3) 200 g of silica microsphere (130-150 meshes) having a particle size of 0.10-0.12 mm was added to a fluid-bed coater with a bottom spray gun. The silica microsphere was coated with the pharmaceutical solution by the bottom spray gun. The blowing frequency was 15-25 Hz, the inlet temperature was 40-60° C., the diameter of the spraying nozzle was 1.0 mm, the atomization pressure was 0.15-0.20 megapascal, and the pumping speed of peristaltic pump was 3-15 rpm. When the silica microsphere temperature reached 30-50° C., start the peristaltic pump to pump the pharmaceutical solution. After the coating was finished, stop the peristaltic pump. 10-30 min later, the silica microsphere was taken out, placed in a 50° C. air dry oven for 2-6 hours, sieved, to yield a lincomycin-containing pharmaceutical microsphere.

### 2. Preparation of Sustained-Release Microsphere of Lincomycin

2.1) Coating an Isolation Layer on the Lincomycin-Containing Pharmaceutical Microsphere

20 g of HPMC was added to 400 mL of purified water, stirred for dissolution, to yield a clear solution.

The lincomycin-containing pharmaceutical microsphere in prescription dosage was added to a fluid-bed coater with a bottom spray gun. The pharmaceutical microsphere was coated with the clear solution by the bottom spray gun. The blowing frequency was 15-25 Hz, the inlet temperature was 40-60° C., the diameter of the spraying nozzle was 1.0 mm, the atomization pressure was 0.15-0.20 megapascal, and the pumping speed of peristaltic pump was 3-15 rpm. When the silica microsphere temperature reached 40° C., start the peristaltic pump to pump the clear solution. After the coating was finished, stop the peristaltic pump. 10-30 min later, the pharmaceutical microsphere was taken out, placed in a 50° C. air dry oven for 2-6 hours, sieved, to yield a lincomycin-containing pharmaceutical microsphere comprising an isolation layer.

2.2) Preparation of Sustained-Release Microsphere of Lincomycin

Preparation of a Sustained-Release Solution

50 g of eudragit RS100, 5 g of triethyl citrate, and 400 mL of 90% alcohol solution were mixed, followed by addition of 15 g of talcum powder, to yield a sustained-release solution.

Preparation of a Sustained-Release Pharmaceutical Microsphere

The lincomycin-containing pharmaceutical microsphere comprising an isolation layer was added to a fluid-bed coater with a bottom spray gun. The pharmaceutical microsphere was coated with the sustained-release solution by the bottom spray gun. The blowing frequency was 15-25 Hz, the inlet temperature was 40-50° C., the diameter of the spraying nozzle was 1.0 mm, the atomization pressure was 0.15-0.20 megapascal, and the pumping speed of peristaltic pump was 3-15 rpm. When the silica microsphere temperature reached 40° C., start the peristaltic pump to pump the clear solution. After the coating was finished, stop the peristaltic pump. 10-30 min later, the pharmaceutical microsphere was taken out, placed in a 50° C. air dry oven for 2-6 hours, sieved, to yield a sustained-release lincomycin-containing pharmaceutical microsphere.

### 3. Preparation of Lincomycin Dry Suspension

The sustained-release lincomycin-containing pharmaceutical microsphere, 8 g of talcum powder, 10 g of mannitol, 15 g of fruit flavor, and 15 g of sodium benzoate were evenly mixed, qualified, packaged, to yield lincomycin dry suspension.

### Example 7: Preparation of Enteric-Coated Capsule of Clarithromycin

### 1. Preparation of Clarithromycin-Containing Pharmaceutical Microsphere

1.1) Materials: 300 g of silica microsphere having a particle size of 0.10-0.12 mm, 250 g of clarithromycin (10 μm), 60 g of hydroxypropyl cellulose, 12 g of talcum powder, and 2200 mL of 60% alcohol.

1.2) Preparation of pharmaceutical solution: 50 g of hydroxypropyl cellulose was added to 2000 mL of 60% alcohol, stirred, and dissolved to yield a clear solution. 250 g of clarithromycin was added to the clear solution, stirred for an hour, followed by addition of 10 g of talcum powder, stirred for 15 min, to yield a pharmaceutical solution.

1.3) 300 g of silica microsphere (130-150 meshes) having a particle size of 0.10-0.12 mm was added to a fluid-bed coater with a side spray gun. The silica microsphere was coated with the pharmaceutical solution by the side spray gun. The rotation speed of the coater was 100-200 rpm. The slit width was 2-5 mm. The blowing frequency was 15-25 Hz, the inlet temperature was 40-60° C., the diameter of the spraying nozzle was 1.0 mm, the atomization pressure was 0.15-0.20 megapascal, and the pumping speed of peristaltic pump was 3-15 rpm. Start the peristaltic pump to pump the pharmaceutical solution. After the coating was finished, stop the peristaltic pump. 10 min later, the silica microsphere was taken out, placed in a 50° C. air dry oven for 2-6 hours, sieved, to yield a clarithromycin-containing pharmaceutical microsphere.

### 2. Preparation of Enteric-Coated Microsphere of Clarithromycin

2.1) Coating an Isolation Layer on the Clarithromycin-Containing Pharmaceutical Microsphere

25 g of HPMC was added to 500 mL of purified water, stirred for dissolution, followed by addition of 5 g of talcum powder, to yield a clear solution.

The clarithromycin-containing pharmaceutical microsphere in prescription dosage was added to a fluid-bed coater with a bottom spray gun. The pharmaceutical microsphere was coated with the clear solution by the bottom spray gun. The blowing frequency was 15-25 Hz, the inlet temperature was 40-50° C., the diameter of the spraying nozzle was 1.0 mm, the atomization pressure was 0.15-0.20 megapascal, and the pumping speed of peristaltic pump was 3-15 rpm. When the pharmaceutical microsphere reached 40° C., start the peristaltic pump to pump the clear solution. After the coating was finished, stop the peristaltic pump. 10-30 min later, the pharmaceutical microsphere was taken out, placed in a 50° C. air dry oven for 2-6 hours, sieved, to yield a clarithromycin-containing pharmaceutical microsphere comprising an isolation layer.

2.2) Preparation of Enteric-Coated Microsphere of Clarithromycin

Preparation of an Enteric-Coated Solution

80 g of eudragit L100-55, 8 g of triethyl citrate, and 1000 mL of 95% alcohol solution were mixed, followed by addition of 13 g of talcum powder, to yield an enteric-coated solution.

Preparation of an Enteric-Coated Pharmaceutical Microsphere of Clarithromycin

The clarithromycin-containing pharmaceutical microsphere comprising an isolation layer was added to a fluid-bed coater. The blowing frequency was 15-25 Hz, the inlet temperature was 40-50° C., the diameter of the spraying nozzle was 1.0 mm, the atomization pressure was 0.15-0.20 megapascal, and the pumping speed of peristaltic pump was 3-15 rpm. When the silica microsphere temperature reached 40° C., start the peristaltic pump to pump the enteric-coated solution. After the coating was finished, stop the peristaltic pump. 10-30 min later, the pharmaceutical microsphere was taken out, placed in a 50° C. air dry oven for 2-6 hours, sieved using 75 and 110 meshes sieves, to yield an enteric-coated clarithromycin-containing pharmaceutical microsphere.

### 3. Preparation of Enteric-Coated Capsules of Clarithromycin

The content of clarithromycin in the enteric-coated clarithromycin-containing pharmaceutical microsphere was determined. According to the results, the pharmaceutical microsphere and corresponding additives were mixed, packaged to produce the enteric-coated capsules of clarithromycin.

The silica microsphere mentioned in the aforesaid examples can be prepared through various methods. Table 1 lists the sources of the silica microsphere and the advantages and disadvantages thereof.

TABLE 1
Sources of the silica microsphere and the advantages and disadvantages thereof

| | Items | | |
|---|---|---|---|
| Sources | Production process | Advantages | Disadvantages |
| Powder method | High purity silica particles are | Hard glass | Long production |
| | crushed, screened, heated and | microspheres can | cycle, low |
| | melted to form solid glass | be prepared, the | output, high cost, |
| | microspheres. | particle size is | high energy |
| | | easy to control, | consumption. |
| | | high yield. | |
| Centrifugal | High purity silica particles are | Short production | The particle size |
| granulation | crushed, and pelleted in a | cycle, high | is not easy to |
| | centrifugal granulator in the | production | control, and the |
| | presence of adhesives to yield | efficiency. | hardness, |
| | glass microspheres. | | brittleness and |
| | | | bulk density of |
| | | | glass pellets are |
| | | | low. |
| Fusion method | The glass complex is melted into a | Low cost, high | Particle size is |
| | glass solution, which is sprayed by | yield. | not easy to |
| | high speed airflow to yield glass | | control, resulting |
| | microspheres. | | in glass fibers or |
| | | | tailed |
| | | | microspheres. |
| Flame floating | The high purity silica is crushed, | Hard glass | — |
| method | put into a beading furnace, melted | microspheres can | |
| | by high temperature air stream, to | be prepared, with | |
| | yield glass microspheres. To | controllable | |
| | prevent the adhesion between the | particle size, | |
| | microspheres, the silica particles | high yield, low | |
| | are in the floating state in the bead | cost, short | |
| | formation process. | production cycle. | |
| Grinding method | High purity silicon dioxide is | Hard glass | — |
| | crushed, put into a beading | microspheres can | |
| | furnace, and ground at high | be prepared, with | |
| | temperature to prepare glass | controllable | |
| | microspheres. | particle size, | |
| | | high yield, high | |
| | | production | |
| | | efficiency, low | |
| | | cost, short | |
| | | production cycle. | |

The experimental results show that the microspheres prepared by the grinding method have the greatest advantages. The glass microspheres prepared by the method have the advantages of uniform particle size, high yield, and low cost.

Table 2 shows the parameters of conventional blank pellets and the pharmaceutical microsphere of silica as described in the disclosure.

TABLE 2
Comparison of conventional blank pharmaceutical pellets with the
pharmaceutical microsphere of the disclosure

| | Index | | | |
|---|---|---|---|---|
| | | Bulk | | |
| | Roundness | density | Rigidity | |
| Products | (°) | (g/cm³) | (%) | Compatibility |
| Sugar type blank | 12.5 | 0.62 | 0.7 | Tends to react |
| pellets | | | | with certain |
| | | | | active |
| | | | | ingredients |
| Starch type blank | 13.1 | 0.61 | 0.9 | Tends to react |
| pellets | | | | with certain |
| | | | | active |
| | | | | ingredients |
| Sugar-starch type | 12.3 | 0.61 | 0.7 | Tends to react |
| blank pellets | | | | with certain |
| | | | | active |
| | | | | ingredients |
| Microcrystalline | 11.2 | 0.64 | 0.7 | Tends to react |
| cellulose blank pellets | | | | with certain |
| | | | | active |
| | | | | ingredients |
| Pharmaceutical | 10.9 | 1.35 | 0.2 | Not react with |
| microsphere of silica | | | | certain active |
| | | | | ingredients |

The roundness is Table 2 is represented by one plane critical stability, which can characterize the roundness of particles in terms of the angle necessary to tilt a plane such that the particles tend to roll.

It will be obvious to those skilled in the art that changes and modifications may be made, and therefore, the aim in the appended claims is to cover all such changes and modifications.

What is claimed is:

1. A method, comprising adding a silica microsphere to an acid, stirring, adding an alkali to neutralize the acid until a pH value of a mixture of the acid and the alkali is 6-7, filtering and collecting a resulting product, washing, drying, and sieving, to yield a pharmaceutical microsphere.

2. The method of claim 1, further comprising heating glass powders in a beading furnace to a temperature of 600-1200° C., and then cooling the glass powders to a temperature of 100-300° C., to yield the silica microsphere.

3. The method of claim 1, further comprising grinding a rock crystal comprising at least 90.0 wt. % of silica to yield the silica microsphere.

4. The method of claim 1, wherein the acid is selected from sulfuric acid, hydrochloric acid, phosphoric acid, nitric acid, or a mixture thereof, and has a concentration of 1-10 wt. %; and the base is selected from sodium hydroxide, potassium hydroxide, or a mixture thereof.

5. The method of claim 1, comprising: preparing the silica microsphere in a beading furnace or grinding a rock crystal to yield the silica microsphere, adding the silica microsphere to a 5-10% hydrochloric acid solution, stirring for 1-2 hours, allowing for standing, removing a supernatant, adding a 1-5 mol/L sodium hydroxide aqueous solution to neutralize the hydrochloric acid solution until a pH value of a resulting mixture is 7, filtering and collecting the resulting product, washing with purified water, drying, and sieving, to yield the pharmaceutical microsphere.

6. The method of claim 1, comprising: preparing the silica microsphere in a beading furnace or grinding a rock crystal comprising 99.0 wt. % of silica to yield the silica microsphere, adding the silica microsphere to a 10% hydrochloric acid solution, stirring for 1 hour, allowing for standing, removing a supernatant, adding a 1 mol/L sodium hydroxide aqueous solution to neutralize the hydrochloric acid solution until a pH value thereof is 7, filtering and collecting the resulting product, washing with purified water, drying, and sieving, to yield a pharmaceutical microsphere.

7. The method of claim 1, comprising: preparing the silica microsphere in a beading furnace or grinding a rock crystal comprising 99.9 wt. % of silica to yield the silica microsphere having 120 meshes, adding the silica microsphere to a 10% hydrochloric acid solution, stirring for 1 hour, allowing for standing, removing a supernatant, adding a 1 mol/L sodium hydroxide aqueous solution to neutralize the hydrochloric acid solution until a pH value thereof is 7, filtering and collecting the resulting product, washing with purified water, drying, and sieving, to yield a pharmaceutical microsphere having 120 meshes.

8. The method of claim 1, wherein a particle size of the pharmaceutical microsphere ranges from 40 to 250 μm; a bulk density of the pharmaceutical microsphere ranges from 1.2 to 1.6 g/cm³; a roundness of the pharmaceutical microsphere is between 10-12°.

9. The method of claim 8, wherein the particle size of the pharmaceutical microsphere ranges from 50 to 100 μm.

10. A method of preparing a pharmaceutical preparation, the method comprising:
adding a silica microsphere to an acid, stirring, adding an alkali to neutralize the acid until a pH value of a mixture of the acid and the alkali is 6-7, filtering and collecting a resulting product, washing, drying, and sieving, to yield a pharmaceutical microsphere, and coating the pharmaceutical microsphere.

11. The method of claim 10, wherein the pharmaceutical preparation has a particle size of no more than 150 μm.

12. The method of claim 10, wherein the pharmaceutical microsphere is coated by an isolation layer, a taste-masking layer, a sustained-release layer, or a combination thereof.
